(19) United States
(12) Patent Application Publication
(10) Pub. No.: US 2002/0198533 A1
(43) Pub. Date: Dec. 26, 2002

(54) Multipositional intervertebral instrument guide

(76) Inventors: Fred Geisler, Evanston, IL (US); Michael Foster, Palo Alto, CA (US); Daniel Bass, El Granada, CA (US); Thomas Wiedenmaier, San Carlos, CA (US); Terry Johnston, Redwood City, CA (US)

Correspondence Address: Kimberly Diliberti, Paralegal, Allegiance Corporation, 1430 Waukegan Road, McGaw Park, IL, 60085, US

(21) Appl. No.: 10/116,264

(22) Filed: Apr. 3, 2002

Related U.S. Application Data
(60) Provisional application No. 60/284,395, filed on Apr. 17, 2001.

(51) Int. Cl. A61B 17/17

(52) U.S. Cl. 606/96

(57) ABSTRACT

The invention disclosed herein relates to a site preparation guide for use in conjunction with intervertebral implants. In particular, the site preparation guide contains a hollow body, lumen and interior surface adapted to accommodate and control the positioning of a surgical tissue removal device during the creation of the implant site. The site preparation guide of the invention can further include side openings through the body of the guide to accommodate and control the positioning of a surgical drill, an integrated intervertebral distractor, and a distal endplate which serves as a drilling template for an implant having a corresponding endplate. The site preparation guide is a unitary, one-piece, sterilizable device. Also disclosed herein is a method of preparing an orthopedic intervertebral site using the site preparation guide of the invention and surgical kit including the guide.

### Related Application Data

This application is based on provisional patent application Ser. No. 60/284,395 filed Apr. 17, 2001.

### FIELD OF THE INVENTION

The invention herein relates to orthopedic surgery of the spine. In particular, the invention pertains to a medical device for preparing an intervertebral surgical site for receipt of an intervertebral implant.

### BACKGROUND OF THE INVENTION

The human spinal column contains a series of subgroups of vertebrae which include the cervical, thoracic, lumbar, sacral and coccygeal vertebrae. Certain intervertebral joints, including the cervical vertebrae, contain an intervertebral disc positioned between the endplates of two adjacent vertebrae. One of the primary functions of the intervertebral disc is to absorb the vertical forces exerted on the spine.

An intervertebral disc is composed of two main portions—the annulus (or annulus fibrosis) and the nucleus (or nucleus pulposus). The annulus is composed of relatively rigid cartilage and functions in part to contain the softer, more flexible nucleus pulposus. The maintenance of the respective functions of each of the nucleus and annulus is essential to normal spinal motion and operation. Damage or disease which affects the structural integrity of the intervertebral disc such as the atrophy or rupturing of the disc, often requires surgery to repair or restore the joint.

One surgical technique involves the insertion of spinal implants into the intervertebral space to restore the space between two adjacent vertebrae. A variety of such implants are known in the art and have a variety of designs, configurations and surface topography. Such implants can be cylindrical, cuboid or trapezoid in shape, solid or hollow, and contain a variety of fixation or securing features. The various configurations and shapes of implants present challenges in designing surgical equipment which is adapted to operate in cooperation with the implant.

Insertion, placement and fixation of a spinal implant between adjacent vertebrae requires precise surgical preparation of the implant site. Generally, the creation of an opening or chamber into which the implant will be placed is necessary. Given the surrounding neurological tissue, it is critical to avoid unnecessary damage to surrounding tissue throughout the procedure. Surgical instruments used in such procedures, such as arthroscopic knives, laser techniques, burrs, rasps, and the like, must be precisely controlled during such procedures. Two general approaches are possible for intervertebral disc repair—posterior and anterior approaches. The posterior approach involves access to the disc space from the area proximal to the spinal cord. The anterior approach, however, involves the access of the intervertebral space from the side of the vertebral column opposite the spinal cord. The anterior approach offers the advantages of reducing the likelihood of damage to the central nervous system and peripheral nerves. Surgical instrumentation for the anterior approach, however, must accommodate the anatomical obstacles involved.

In addition to the difficulties associated with controlling the position and movement of such instruments, the implant site must be prepared in accordance with the requirements, e.g., dimensions, of the implant to be positioned. Certain types of implants which involve unusual shapes or configurations and/or structural fixation features, such as endplates with screwholes, present additional difficulties in the preparation of the site.

Various site preparation instruments associated with spinal implants are known in the art. Yonemura et al. U.S. Pat. No. 6,156,040 discloses an apparatus for implanting a spinal implant using a drill guide. The apparatus also includes a drill tube with distraction paddles. This apparatus, however, includes the use of an external centering guide and allows for a single site preparation position. Kuslich et al. U.S. Pat. No. 5,489,307 discloses an intervertebral site preparation device which uses a temporary distractor in conjunction with a bore guide. The bore guide, however, is designed to create cylindrical bores for cylindrical implants, and further only allows for a single position for the boring instrument.

Other devices include those of Michelson U.S. Pat. Nos. 5,797,909, 6,096,038 and 6,080,155, which disclose apparatuses for inserting spinal implants comprising an outer sleeve through which both the instruments to remove bone and disc tissue are inserted and subsequently through which the implants are to be delivered to the site. The devices also contain distracting extensions on the outer sleeve to distract the intervertebral joint upon positioning of the device. Because the outer sleeve of these devices must also deliver the implant, the extent of possible modification of the interior surface for tissue removal devices is limited since the sleeve must have a configuration which accommodates the larger dimensions of the implant. Accordingly, the ability to precisely control the positioning of the site preparation instrumentation used in conjunction therewith is compromised.

There exists a need in the spinal surgery field for improved site preparation tolls for spinal implants which improve surgical precision of the procedure. Of further interest in the field would be a site preparation instrument which is adapted to accommodate and control the positioning of various instruments in a manner which prepares the site in accordance with the specific attributes of the implant to be placed therein.

### SUMMARY OF THE INVENTION

The invention provides for a site preparation guide for intervertebral implants which contains structural features uniquely designed to cooperate with and optimize the function of surgical instruments associated with site preparation for spinal implants. In particular, the site preparation guide of the invention includes structural features which: a) accommodate and facilitate the control and positioning of surgical tissue removal devices such as rasps and burrs; b) permit such control over multiple incremental positions during the procedure; and preferably further includes c) side openings in the device to position and control a surgical drill; d) and integrated intervertebral distractor; and e) a distal endplate which serves as a drilling template for the implant to be placed and secured. Surprisingly, it has been discovered that all of the before-mentioned features can be accomplished with a simplified, unitary, integrated, sterilizable device. Because the site preparation guide of the invention is not used to deliver the implant to the site, the device can be adapted in an advantageous instrument-specific manner and can be configured to produce non-cylindrical implant sites thereby facilitating the precise creation of the implant site by the surgeon. The invention includes a site preparation guide, an intervertebral site preparation kit, and a method of preparing an intervertebral site using the guide.

The invention provides for a site preparation guide for an intervertebral implant comprising: an elongate hollow body having proximal and distal ends; a single contiguous lumen running through the entire length of said body providing an interior surface adapted to accommodate and control the position of a surgical instrument inserted therethrough; wherein the interior surface has at least two substantially parallel adjacent longitudinal ridges on the same side of the surface corresponding to at least three incremental positions for said surgical instrument. In a preferred embodiment, the site preparation guide comprises diagonally-oriented openings adapted for placement of a surgical drill in diagonal alignment therethrough so that when said drill is inserted through the openings the distal end of the drill exits through the distal portion of the guide. In an even more preferred embodiment, the distal end of the hollow body of the site preparation guide of the invention contains an endplate adapted to simultaneously contact the exterior surfaces of two adjacent vertebrae. In a most preferred embodiment, the distal end of the site preparation guide includes at least one distraction tang extending distally therefrom and adapted for insertion into the intervertebral space.

The invention also provides for a method of preparing an intervertebral site in a patient comprising: a) accessing the intervertebral site; b) inserting and positioning a site preparation guide having an elongate hollow body having proximal and distal ends; a single contiguous lumen running through the entire length of said body providing an interior surface adapted to accommodate and control the position of surgical instruments inserted therethrough; wherein the interior surface has at least two substantially parallel adjacent longitudinal ridges on the same side of the surface corresponding to at least three incremental positions for said surgical instruments; and wherein the distal end of said hollow body further comprises an end plate adapted to simultaneously contact the exterior surfaces of two adjacent vertebrae; c) fixing said preparation guide to the site by drilling through the exterior surfaces of two adjacent vertebrae by inserting a surgical drill through diagonally-oriented openings through the guide body and end plate guide holes and inserting a fixation device into the vertebrae thereby securing said endplate thereto; d) inserting a surgical instrument through the site preparation guide lumen and positioning said instrument along said ridges; and e) removing an amount of tissue from the site sufficient to accommodate an intervertebral implant.

The invention further provides for an intervertebral site preparation kit comprising: a site preparation guide having an elongate hollow body having proximal and distal ends; a single contiguous lumen running through the entire length of said body providing an interior surface adapted to accommodate and control the position of a surgical instrument inserted therethrough; wherein the interior surface has at least two substantially parallel adjacent longitudinal ridges on the same side of the surface corresponding to at least three incremental positions for said instrument; in combination with at least one intervertebral implant. The kit can further include at least one additional surgical instrument selected from the group consisting of burrs, rasps, drills, screws, and combinations thereof.

### BRIEF DESCRIPTION OF THE DRAWINGS

The following descriptions, when taken together with the accompanying drawings and numerical designations which remain consistent throughout, illustrate the objects and advantages of the invention.

FIG. 1 is an angled perspective view of the site preparation guide according to one embodiment of the invention.

FIG. 2 is a top plan view of the site preparation guide according to one embodiment of the invention.

FIG. 3 is an end view of the proximal end of the site preparation guide with outlines illustrating several incremental positions according to one embodiment of the invention.

FIG. 4 is an end view of the distal end of the preparation guide with outlines illustrating several incremental positions according to one embodiment of the invention.

FIG. 5 is an angled perspective view of the distal portion of the site preparation guide according to one embodiment of the invention.

FIG. 6 is an end view of the distal end of the site preparation guide according to one embodiment of the invention.

FIGS. 7A and 7B are together angled top view perspectives of one embodiment of the site preparation guide of the invention together with an intervertebral implant showing the cooperating alignment of the guide holes of the endplate and fixation holes of the implant.

FIG. 8 is a side view of the site preparation guide showing the diagonal alignment of the diagonally-oriented side holes in the body of the guide and through an endplate guide hole according to one embodiment of the invention.

### DETAILED DESCRIPTION OF THE DRAWINGS

As used herein, the term “site” is meant to refer to the intended location of spinal surgery. In the context of the site preparation guide of the invention, the term is meant to refer to that portion of the spine, vertebrae, and intervertebral disc space to be accessed and prepared for receipt of an intervertebral implant, for example.

The term “intervertebral” as used herein is meant to define the region between and including the opposing endplates of two adjacent vertebrae of the spine and immediately surrounding tissues.

The term “surgical instrument” as used herein is meant to include any surgical device or instrument which is useful for, or adapted for use in, surgical procedures in the spine, and which can be used in association with the site preparation guide of the invention. Suitable surgical instruments include, but are not limited to, tissue removal devices, such as trocars, burrs, rasps, and the like.

The term “substantially parallel” as used to describe the positions of the longitudinal ridges is meant to describe the relative positioning of the ridges throughout the length of the interior surface in which they are present. The term is meant to include slight deviations from absolute parallelism provided the effect is that the positioning of an orthopedic instrument residing within the lumen can be controlled during the procedure.

The term “incremental” when referring to positioning of an instrument within the device is meant to indicate the availability of different spatially predetermined locations. The term is not intended to be limited to immediately adjacent positional relationships.

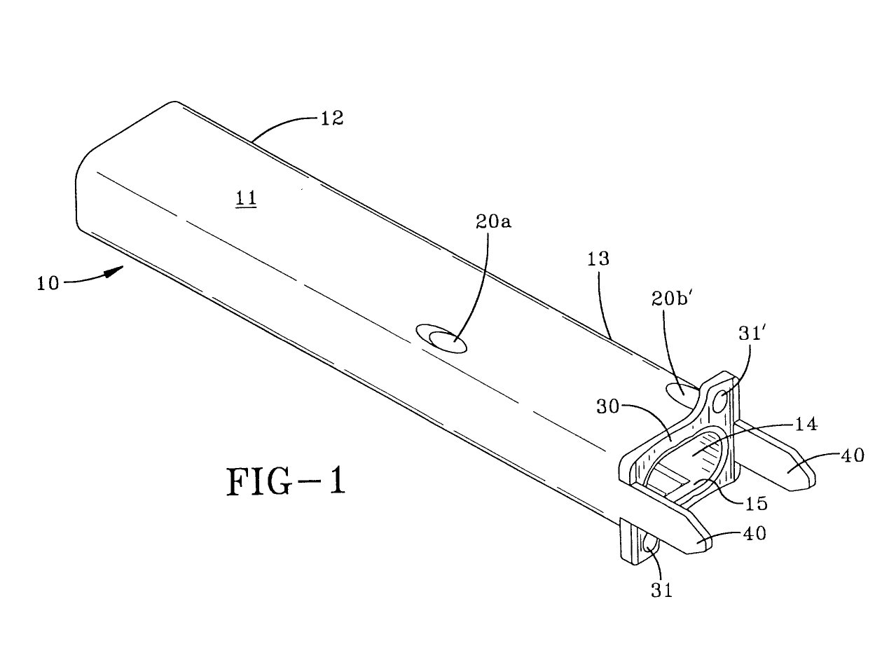

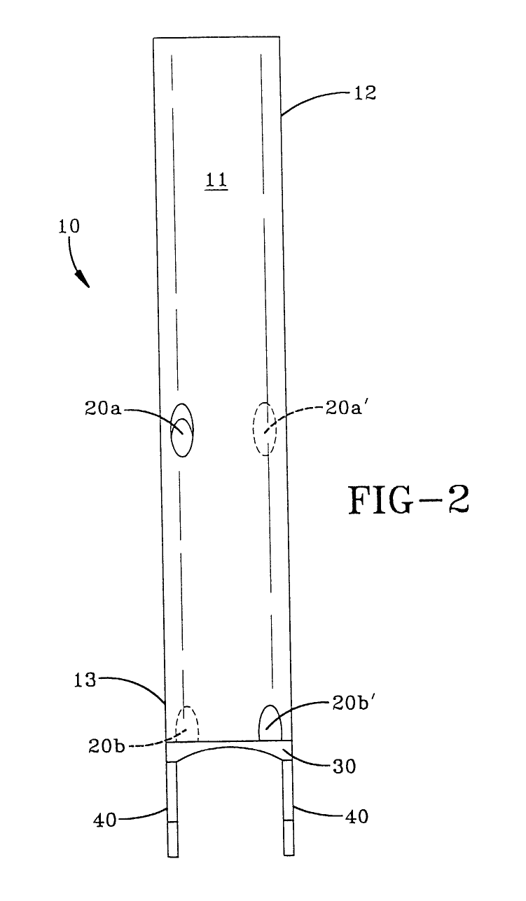

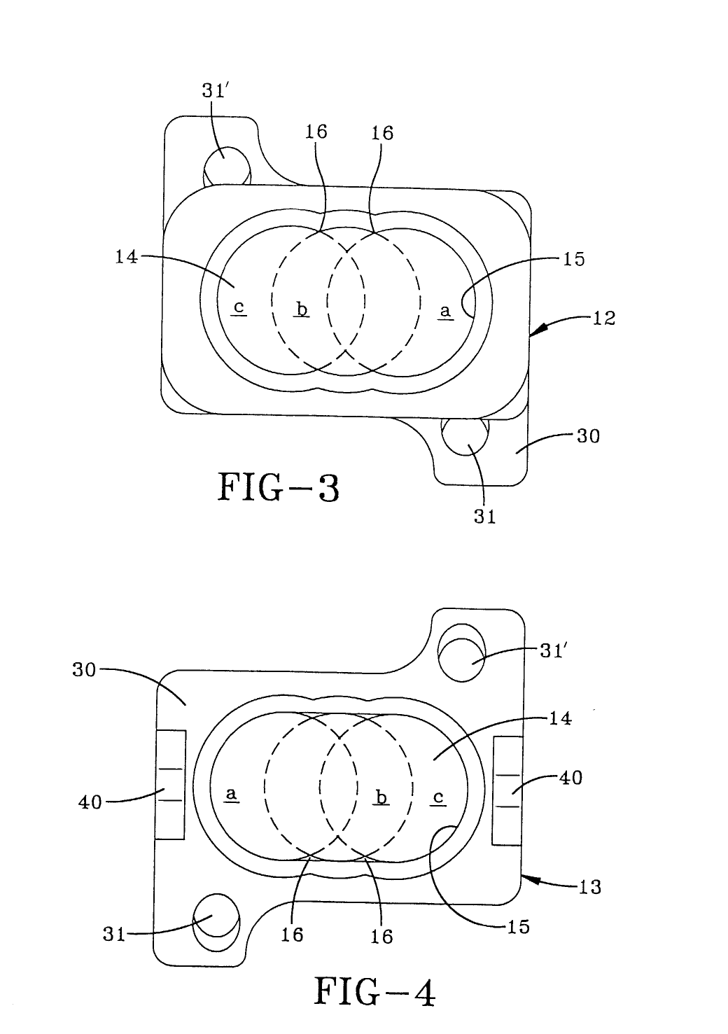

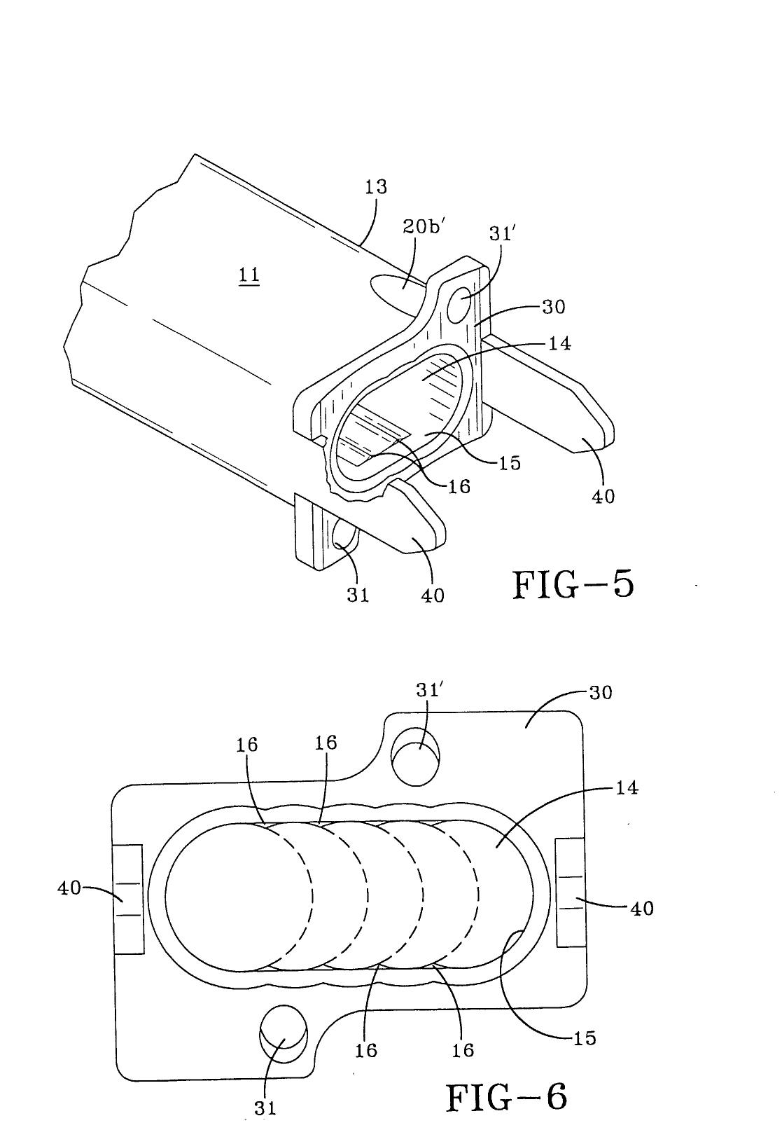

Referring to FIG. 1, the site preparation guide 10 of the invention generally comprises an elongate hollow body 11 having proximal and distal ends 12 and 13 respectively; a single contiguous lumen 14 running through the entire length of said body providing an interior surface 15 adapted to accommodate and control the position of a surgical instrument (not shown) inserted therethrough; wherein the interior surface 15 has at least two substantially parallel adjacent longitudinal ridges 16 (see FIGS. 3 through 6) on the same side of the surface corresponding to at least three incremental positions (shown as a, b and c in FIGS. 3 and 4) for said surgical instrument. The elongate hollow body portion 11 can vary in length and width provided the overall dimensions are sufficient to afford the device with the length and width compatible with its usage, e.g., a length sufficient to permit access to the implant site to prepare for the implant while avoiding interference by surrounding tissues, and a width sufficient to contain the desired structural features.

The single contiguous lumen 14 runs through the entire length of the device uninterrupted. The dimensions of the lumen 14 and interior surface 15 are coordinated to provide a guide which accommodates the surgical instruments (e.g., tissue removal device) with which it is to be used. The dimensions of the lumen 14 and interior surface 15 of the guide are also adapted to prepare an implant site having substantially the same overall dimensions as the portion of the implant to be inserted into the disc space. The accommodation and control of the position of the surgical instrument is accomplished by virtue of the fit of the instrument within the guide. The fit generally permits free longitudinal movement of the instrument along the guide, while the lateral movement of the instrument can be controlled, either partially or completely, by the interior surface structure and configuration. According to the invention, the interior surface 15 comprises at least two substantially parallel adjacent longitudinal ridges 16 on the same side of the surface corresponding to at least three incremental positions for said surgical instrument. Incremental positions are illustrated in FIGS. 3, 4 and 6 as dotted circular lines and designated as a, b and c in FIGS. 3 and 4. The longitudinal body of the surgical instrument, therefore, can rest in alignment between these ridges during its operation. Movement from one inter-ridge location to the next affords incremental positioning of the instrument both within the guide and beyond the distal end of the guide thereby affording precise removal of the bone and disc tissue as needed.

The internal surface 15 of the guide comprises substantially parallel longitudinal ridges 16 located at lest on a single side of the interior surface of the guide. In one embodiment and as shown in FIGS. 3 through 6, however, the lumen 14 and interior surface 15 together can provide a generally ovoid configuration from an end perspective, and the interior surface comprises corresponding pairs of opposing substantially parallel longitudinal ridges.

The dimensions of the ridges can vary in accordance with extent of control desired over the positioning of the instrument. For example, a shallower ridge would more readily permit the repositioning of the surgical instrument whereas a more prominent ridge would increasingly restrict the movement of the instrument within the guide. The site preparation guide of the invention can contain a combination of varying ridge (and groove) dimensions. Furthermore, the longitudinal ridges need not be present throughout the entire length of the interior surface of the guide.

The number of ridges can vary in accordance with the desired configuration of the implant site to be created and/or the dimensions of the surgical instrument. The site preparation guide of the invention can have an interior surface having from 2 to about 7 pairs of opposing ridges. In one embodiment, the guide comprises 4 to about 7 pairs of opposing ridges. Furthermore, the ridges need not be strictly parallel and can deviate somewhat to permit positioning of the surgical instrument and a slight angle relative to another position. This provides advantages with respect to implants which have varied width or tapered configurations.

Because the site preparation guide 10 of the invention is not used to deliver the implant to the site, the lumen 14 and internal surface 15 of the site preparation guide can be modified to permit the creation of a variety of possible implant sites specifically adapted for the specific shape and configuration of the implant. The site preparation guide therefore permits the creation of implant site configurations other than the conventional cylindrical site of the cylindrical implant. For example, the site preparation guide can comprise a lumen and interior surface geometry which, when used in combination with an orthopedic tissue removal instrument, can produce an elongated ovoid, square or rectangular implant site shape.

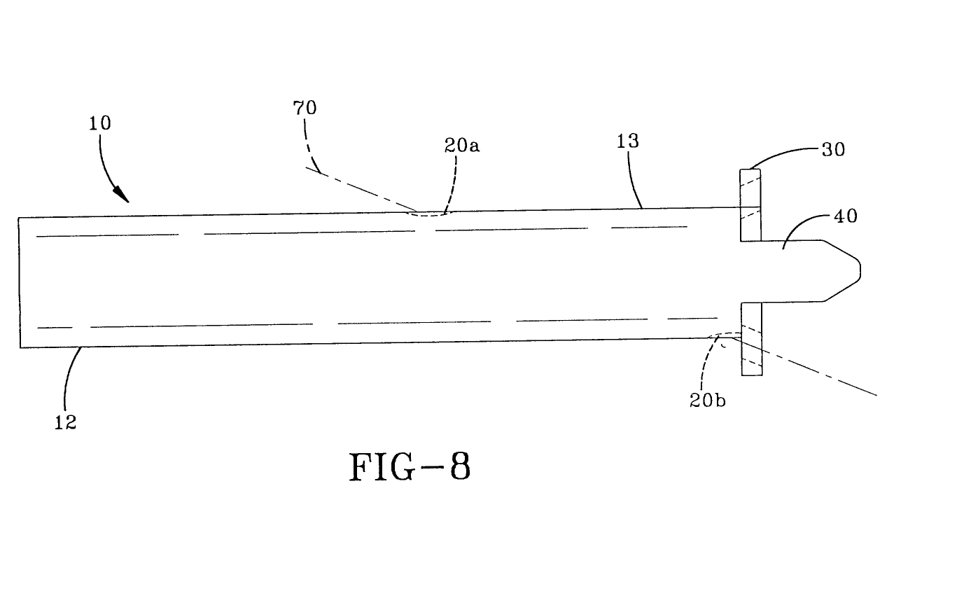

In a preferred embodiment, the site preparation guide of the invention comprises diagonally-oriented openings 20 adapted for placement of a surgical drill in diagonal alignment therethrough so that when said drill is inserted through the openings, the distal end of the drill exits through the distal position 13 of the guide. The number and positioning of these openings can vary provided the orientation of each pair is such that the lateral movement of the drill is restricted so as to avoid unnecessary damage to the surrounding tissues. Referring to FIG. 8, the diagonal orientation refers tot he resulting position of the surgical drill when inserted through the pair of openings relative to the guide lumen 14. The position of the drill is symbolically represented as a dotted line in FIG. 8. Such positioning permits the operation of the drill in a relatively longitudinal alignment with the site preparation guide and controls the positioning of the drill at two points (i.e., proximally and distally).

The diagonally-oriented openings 20 consist of at least one proximal opening 20a and at least one distal opening 20b through the body 11 and are arranged so that each opening of a given pair are located on substantially opposing sides such that one pair of openings will operate with the drill to create a single drill site on the exterior surface of a vertebra. For purposes of illustration, the proximally positioned diagonally-oriented openings are referred to as 20a and 20a′ and the distal diagonally-oriented openings as 20b and 20b′. It will be understood, however, that each pair of diagonally-oriented openings 20a and 20b, or 20a′ and 20b′, need not be on the extreme opposite location of the guide body. The number of diagonally-oriented pairs of openings can vary as well according to the number of drilled sites on the exterior surface(s) of each vertebra to be created. For example, the site preparation guide adapted to create one drill site on each of two adjacent vertebrae can have two pairs of diagonally-oriented openings wherein each pair permits the creation of a single drill site.

In an even more preferred embodiment of the invention, the distal end of the hollow body of the site preparation guide of the invention contains an endplate 30 adapted to simultaneously contact the exterior surfaces of two adjacent vertebrae. The endplate preferably has a configuration, i.e., height, width, protrusions and recesses, adapted to further more accurately fit onto the surfaces by accommodating the anatomy of the joint. The endplate 30 contains a portion which extends laterally relative to the distal position of the guide body. The endplate need not have any particular overall shape provided it provides the structure suitable for its intended function. In other words, the endplate need not have an overall square-like perimeter as shown in the Figures. The endplate 30 functions to permit the securing of the site preparation guide of the invention into position during the site preparation procedure.

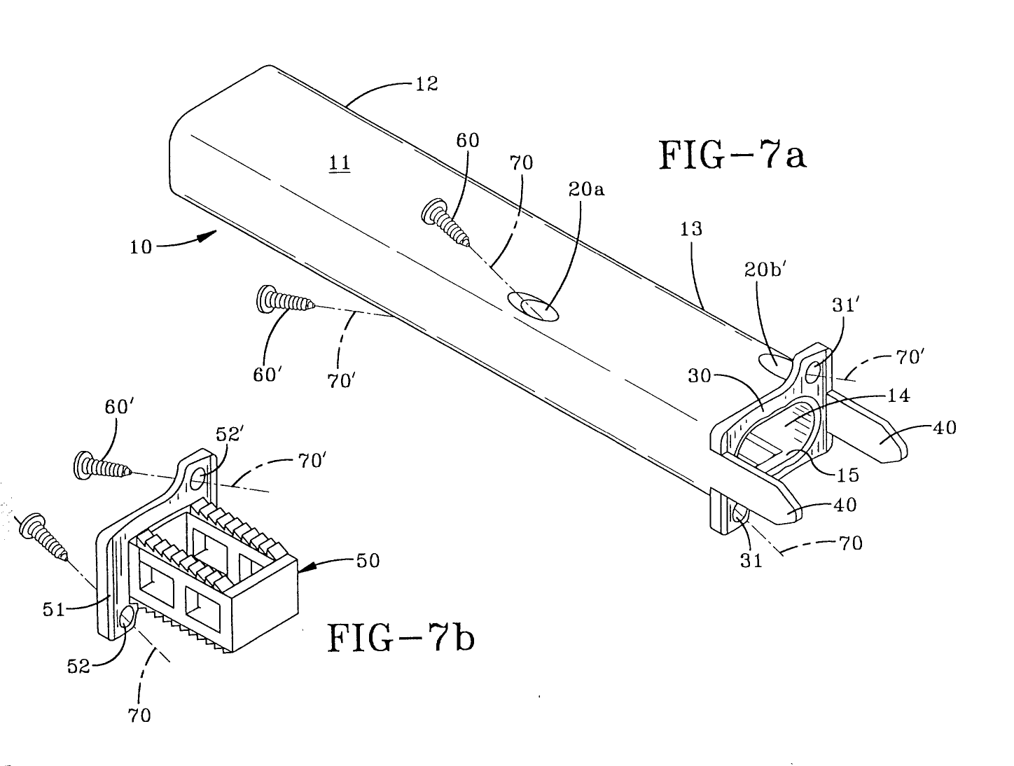

In a preferred endplate embodiment, the endplate further 30 comprises guide holes 31 and 31′ for a surgical drill (not shown) which align with a given pair of diagonally-oriented openings 20a and 20b, and 20a′ and 20b′ respectively, on the site preparation guide body 11. The location, size and configuration of the guide holes 31 correspond to the dimension and desired positioning of the intended fixation device. In accordance with this embodiment, the endplate guide holes 31 and 31′ perform the dual function of securing the site preparation guide to the exterior surface of the vertebrae during the procedure, as well as the function of providing the drilling template to prepare the drill holes for receipt of the fixation device, e.g., screw, to fix the final position of an implant 50. This embodiment is depicted in FIGS. 7A and 7B, which illustrate the relative alignment of the endplate guide holes 31 and 31′ (alignment depicted as lines 70 and 70′) to a pair of screws 60 and 60′ respectively, used to secure a spinal implant 50. Depicted as linear alignment relationships 70 and 70′, the diagonally-oriented openings 20a and 20b, and 20a′ and 20b′, produce an angled approach relative to the endplate 30 as illustrated in FIG. 8. Accordingly, the drill site created in the vertebral body is formed in line with such an angle, and the fixation device (e.g., screws 60) when placed through either the guide holes 31 and 31′ of the endplate 30 of the guide 10, or the fixation holes 52 and 52′ of the endplate 51 of the implant 50, resides at such an angle as well.

The site preparation guide of the invention can be modified so as to be compatible with a variety of spinal implant devices with which it is to be used. One example of an implant which can benefit from the features of the guide is that disclosed by Geisler et. al., U.S. patent application Ser. No. 09/382,808 and issued as U.S. Pat. No. 6,231,610 the entire text of which is incorporated herein by reference.

In a most preferred embodiment, the distal end 13 of the site preparation guide 10 includes at least one distraction tang 40 extending distally therefrom and adapted for insertion into the intervertebral space. The distraction tang can have any configuration provided the height is sufficient to maintain the desired intervertebral space. Typically, the distraction tang will have a height greater than its width, and a length sufficient to reside within the intervertebral space. Preferably and as depicted in the Figures, two distraction tangs 40 are present and are located on opposing sides of the distal end of the guide 10. Ideally, the tip of the distraction tang is shaped to facilitate penetration into the intervertebral disc space by longitudinal force. The tip can be tapered, rounded, beveled, and the like. When distraction tangs are present on the site preparation guide of the invention, the guide simultaneously distracts and maintains intervertebral spacing during the site preparation procedure without interfering with the operation of the associated surgical instruments used.

The site preparation guide of the invention can be composed of any materials having sufficient structural integrity to accommodate the instruments with which the guide is to be used and resist deformation thereby during use. Preferably, the material is also sterilizable or autoclavable as well. Typically, the material is composed of metal or a metallic alloy. Suitable materials for the site preparation guide include, but are not limited to, stainless steel and titanium.

The site preparation guide of the invention can be made using a variety of conventional equipment and techniques known in the orthopedic manufacturing field. Mold and die techniques and equipment can be used, alone or in combination with a variety of shaping tools.

One embodiment of the method of preparing an intervertebral site in accordance with the invention involves accessing the intervertebral site using various conventional orthopedic and/or arthroscopic equipment and instruments. The site preparation guide 10 of the invention is specifically adapted for the anterior approach for intervertebral access and repair. Once the intervertebral site has been accessed, the site preparation guide 10 of the invention is inserted and positioned so that the distal end 13 of the site preparation guide contact the exterior surfaces of the adjacent vertebrae. When distraction tangs 40 are present on the distal end of the guide 10, longitudinal forces exerted on the guide 10 insert the distraction tangs 40 into the intervertebral space thereby distracting the joint and maintaining the intervertebral space throughout the procedure. In the case of the guide having an end plate 30, the end plate 30 simultaneously contacts the exterior surfaces of two adjacent vertebrae. Next, the site preparation guide is fixed or secured to the surrounding vertebral bodies of the adjacent vertebrae by inserting a surgical drill through the diagonally-oriented openings 20a and 20b, and 20a′ and 20b′, through the body 11 of the guide 10 and through the endplate 30 and drilling into the exterior surface of each vertebra. Fixation devices, such as orthopedic screws or nails adapted for use in the spine, are then inserted and secured into the drill site using the appropriate fixation tool thereby securing the distal end of the site preparation guide onto the vertebrae.

After fixation of the guide 10 to the vertebrae, surgical instruments are inserted through the lumen 14 of the site preparation guide 10. Various surgical instruments can be used in conjunction with the surgical procedure. Insofar as the instruments used to create the opening or chamber for the implant site to be inserted, surgical instruments adapted to remove bone and disc tissue are used. To benefit from the site guide of the invention, at least one such instrument is adapted to reside and be retained along the substantially parallel ridges on the interior surface of the guide. Suitable instruments for use with the invention include, but are not limited to, drills, burrs, rasps, and the like. Typically, a burr can be used to remove disc tissue and bone from the implant site. Ultimately, an amount of tissue will be removed from the site sufficient to accommodate an intervertebral implant.

During the tissue removal stage of the procedure, the surgical instrument adapted to remove tissue is repositioned by moving the instrument alongside a different ridge 16. Once positioned and residing therein, the instrument will be aligned at a different incremental position relative to the previous position. The instrument can be positioned into the adjacent position formed by the ridges or any other position offered by the site preparation guide as deemed appropriate by the practitioner. The instrument is thus controlled and stabilized during the tissue removal stage and increases the precision and accuracy of the surgery. Furthermore, the site preparation guide functions overall as a template for the configuration of the opening or chamber for the implant.

After the implant site has been created, the site preparation guide is detached and removed from the implant site. Where a fixation device is removed from the vertebrae by using the same diagonally-oriented openings.

The prepared implant site contains a created opening or chamber which is specifically and precisely adapted to accommodate the corresponding spinal implant 50. Furthermore, in the case of an implant having an endplate 51 with fixation holes 52 and 52′, the prepared implant site contains drill sites which align with the fixation holes 52 and 52′ of the implant such that the site has also prepared the vertebrae for receipt of the fixation device to be used in conjunction with the implant.

The intervertebral site preparation kit of the invention comprises the site preparation guide of the invention in combination with at least one intervertebral implant. The site preparation guide of the kit is one which is compatible with the intervertebral implant with which it is to be used. For example, the kit can include a site preparation guide having an endplate and an implant having an endplate such as that disclosed by Geisler et. al., U.S. patent application Ser. No. 09/382,808 and issued as U.S. Pat. No. 6,231,610. More than one spinal implant can be included in the kit. The site preparation guide of the invention can work in conjunction with any spinal implant which can benefit from the accurate and precise preparation of the intervertebral site.

In addition to the site preparation guide of the invention, the kit of the invention can further include at least one additional surgical instrument typically associated with spinal implant surgery. Suitable surgical instruments which can be included in the kit include, but are not limited to, those selected from the group consisting of burrs, rasps, drills, screws, and combination thereof.

### INDUSTRIAL APPLICABILITY

The invention facilities the surgical preparation of an intervertebral implant site using a simplified, one-piece site preparation guide which is designed to accommodate and control the positioning of surgical and/or orthopedic instruments associated with spinal implantation. Furthermore, the site preparation guide of the invention can be adapted to operate in cooperation with a variety of implant structures and configurations, thereby ensuring compatibility between the guide and a variety of intervertebral implants.

The complete disclosures of all patents, patent applications, and publications are incorporated herein by reference as if each were individually incorporated by reference. The invention has been described with reference to various specific embodiments and techniques. It will be understood by one of ordinary skill, however, that many variations of the embodiments and techniques are possible without departure from either the spirit or scope of the invention as set forth in the claims.

What is claimed is:

1. A site preparation guide for an intervertebral implant comprising:
an elongate hollow body having proximal and distal ends;
a single contiguous lumen running through the entire length of said body providing an interior surface adapted to accommodate and control the position of a surgical instrument inserted therethrough;
wherein the interior surface has at least two substantially parallel adjacent longitudinal ridges on the same side of the surface corresponding to at least three incremental positions for said surgical instrument.

2. The site preparation guide of claim 1 wherein the interior surface comprises from about two to about seven pairs of substantially parallel ridges, each ridge of a pair being positioned on opposing sides of the internal surface.

3. The site preparation guide of claim 2 wherein the interior surface comprises four to seven pairs of substantially parallel ridges.

4. The site preparation guide of claim 1 wherein the body of the guide contains at least one pair of diagonally-oriented openings adapted for placement of a surgical drill in diagonal alignment therethrough so that when said drill is inserted through said pairs of openings, the distal end of the drill exits through the distal portion of the guide on the opposing side.

5. The site preparation guide of claim 4 wherein said pair of diagonally-oriented openings consists of a first distal opening and a second opening positioned proximal thereto on the opposing side of the body.

6. The site preparation guide of claim 1 wherein the distal end of said hollow body further comprises at least one distraction tang extending distally therefrom and adapted for insertion in the intervertebral space.

7. The site preparation guide of claim 6 wherein the distal end of said hollow body comprises a pair of distraction tangs located at opposing sides of the guide.

8. The site preparation guide of claim 1 wherein the dimensions of said surgical instrument cooperate with the dimensions of the lumen and interior surface of the guide in a manner sufficient to retain incremental positioning of said instrument.

9. The site preparation guide of claim 8 wherein the surgical instrument is a burr.

10. A site preparation guide for an intervertebral implant comprising:
an elongate hollow body having proximal and distal ends;
a single contiguous lumen running through the entire length of said body providing an interior surface adapted to accommodate and control the position of a surgical instrument inserted therethrough;
wherein the interior surface has at least two substantially parallel adjacent longitudinal ridges on the same side of the surface corresponding to at least three incremental positions for said surgical instrument; and
wherein the distal end of said hollow body further comprises an end plate adapted to simultaneously contact the exterior surfaces of two adjacent vertebrae.

11. The site preparation device of claim 10 wherein said endplate comprises at least one guide hole through said plate, said guide hole adapted to accommodate a surgical drill.

12. The site preparation device of claim 10 wherein the interior surface comprises from about two to about seven pairs of substantially parallel ridges, each ridge of a pair being positioned on opposing sides of the internal surface.

13. The site preparation guide of claim 12 wherein the interior surface comprises four to seven pairs of substantially parallel ridges.

14. The site preparation guide of claim 11 wherein the body of the guide contains at least one pair of diagonally-oriented openings adapted for placement of a surgical drill in diagonal alignment therethrough so that when said drill is inserted through said pair of openings, the distal end of the drill exits trough the distal portion of the guide on the opposing side and penetrates at least one endplate guide hole.

15. The site preparation guide of claim 14 wherein said pair of diagonally-oriented openings consists of a first distal opening and a second opening positioned proximal thereto on the opposing side of the body.

16. The site preparation guide of claim 10 wherein the distal end of said hollow body further comprises at least one distraction tang extending distally therefrom and adapted for insertion in the intervertebral space.

17. The site preparation guide of claim 16 wherein the distal end of said hollow body comprises a pair of distraction tangs located at opposing sides of the guide.

18. The site preparation guide of claim 20 wherein the dimensions of said surgical instrument cooperate with the dimensions of the lumen and internal surface of the guide in a manner sufficient to retain incremental positions of said instrument.

19. The site preparation guide of claim 18 wherein the orthopedic instrument is a burr.

20. A method of preparing an intervertebral orthopedic site in a patient comprising:
a) accessing the intervertebral site;
b) inserting and positioning a site preparation guide having
i) an elongate hollow body having proximal and distal ends;
ii) a single contiguous lumen running through the entire length of said body providing an interior surface adapted to accommodate and control the position of a surgical instrument inserted therethrough;
wherein the interior surface has at least two substantially parallel adjacent longitudinal ridges on the same side of the surface corresponding to at least three incremental positions for said surgical instrument; and
wherein the distal end of said hollow body further comprises an end plate having guide holes therethrough and adapted to simultaneously contact the exterior surfaces of two adjacent vertebrae;
c) fixing said preparation guide to the site by drilling through the exterior surfaces of two adjacent vertebrae by inserting a surgical drill through diagonally-oriented openings through the guide body and end plate guide holes and inserting a fixation device into the vertebrae thereby securing said endplate thereto;
d) inserting a surgical instrument through the site preparation guide lumen and positioning said instrument along said ridges; and
e) removing an amount of tissue from the site sufficient to accommodate an intervertebral implant.

21. The method of claim 20 wherein said surgical instrument is repositioned within the guide in cooperation with said ridges.

22. The method of claim 20 wherein said surgical instrument is a burr.

23. The method of claim 20 wherein said fixation device is a screw.

24. The method of claim 20 further comprising the steps of:
f) detaching and removing the site preparation guide from the site; and
g) inserting an intervertebral implant into the preparation site, said implant having an endplate with fixation holes positioned in alignment with the holes in the exterior surface of the adjacent vertebrae prepared by step c).

25. An intervertebral site preparation kit comprising:
a) a site preparation guide having an elongate hollow body having proximal and distal ends;
a single contiguous lumen running through the entire length of said body providing an interior surface adapted to accommodate and control the position of a surgical instrument inserted therethrough;
wherein the interior surface has at least two substantially parallel adjacent longitudinal ridges on the same side of the surface corresponding to at least three incremental positions for said surgical instrument in combination with
b) at least on intervertebral implant.

26. The intervertebral site preparation kit of claim 25 further comprising at least one additional surgical instrument selected from the group consisting of burrs, rasps, drills, screws and combinations thereof.
